(19) United States
(12) Patent Application Publication
(10) Pub. No.: US 2015/0017631 A1
(43) Pub. Date: Jan. 15, 2015

(54) REAL-TIME PCR DETECTION OF SEASONAL INFLUENZA H1, H3 and B SUBTYPES

(71) Applicant: VELA OPERATIONS PTE. LTD, Singapore (SG)

(72) Inventors: Xin Zhang, Singapore (SG); Hongliang Qian, Singapore (SG)

(73) Assignee: Vela Operations PTE. LTD, Singapore (SG)

(21) Appl. No.: 14/381,525

(22) PCT Filed: Feb. 28, 2013
(86) PCT No.: PCT/IB2013/051592

§ 371 Date: Aug. 27, 2014

(30) Foreign Application Priority Data
Feb. 29, 2012 (GB) 1203534.1

(51) Int. Cl. C12Q 1/70

(52) U.S. Cl. CPC C12Q 1/70; C12Q 2600/166; C12Q 2600/112
USPC 435/5

(57) ABSTRACT

The present invention relates to assays, diagnostic kits and methods for the simultaneous real-time PCR detection of influenza viruses selected from influenza A, in particular subtypes H1 and/or H3, and/or influenza B.

### TECHNICAL FIELD OF THE INVENTION

The present invention relates to diagnostics of infectious microorganisms, and more particularly to the detection of influenza viruses using real-time PCR.

### BACKGROUND

Influenza virus is an infectious microorganism belonging to the family of Orthomyxoviridae. There are two main types of influenza (flu) virus: Types A and B. The influenza A and B viruses that routinely spread in people (human influenza viruses) are responsible for seasonal flu epidemics each year. Influenza A viruses can be broken down into sub-types depending on the genes that make up the surface proteins. Over the course of a flu season, different types (A & B) and subtypes (influenza A) of influenza circulate and cause illness. Influenza A viruses are divided into subtypes based on two proteins on the surface of the virus: the hemagglutinin (H) and the neuraminidase (N). There are 16 different hemagglutinin subtypes and 9 different neuraminidase subtypes. Influenza A viruses can be further broken down into different strains. Current subtypes of influenza A viruses found in people are influenza A (H1N1) and influenza A (H3N2) viruses. In the spring of 2009, a new influenza A (H1N1) virus emerged to cause illness in people. This virus was very different from regular human influenza A (H1N1) viruses and the new virus caused the first influenza pandemic in more than 40 years. That virus (often called “2009 H1N1”) has now mostly replaced the H1N1 virus that was previously circulating in humans. Influenza B viruses are not divided into subtypes, but can be further broken down into different strains. (cf. www.cdc.gov/flu).

Influenza viruses are enveloped RNA viruses and are capable of infecting the respiratory tract of birds and mammals. Influenza A is the most virulent human influenza pathogen and cause the most severe disease. According to the WHO (Influenza (Seasonal), World Health Organization, April 2009), influenza spreads around the world in seasonal epidemics and results in the deaths of about 250,000 to 500,000 people a year. In pandemic years, this number may rise to millions.

Influenza vaccines are available, but vaccination must be refreshed every year owing to the high mutation rate of the viral RNA resulting in different viral strains predominating each new flu season. Numbers of vaccinated individuals in the total population are frequently not sufficient to prevent epidemics or even pandemics. One of the reasons is that the production of vaccine is currently performed in embryonated eggs, which is time-consuming and may not yield sufficient quantities of vaccine for large epidemics or pandemics, in particular when new virus strain arise during the flu season.

Accordingly, rapid and specific diagnosis of influenza virus nucleic acids is crucial for initial detection, successful outbreak control within hospitals and the community, isolation of patients from others and for directing treatment.

The main object of the present invention is to provide assays, kits, compositions and methods suitable for the detection and/or diagnosis of influenza virus nucleic acids in biological samples, wherein the method is simple, highly specific and suitable for simultaneously detecting the presence or absence of different influenza virus subtypes and strains, respectively.

Accordingly, the present invention relates to a set of nucleic acids, useful for simultaneous detection of influenza A virus subtypes H1 and/or H3 and influenza B virus in a biological sample, the set comprising (i) a first pair of primers and at least one first probe, specific for human influenza A/H1 subtypes; (ii) a second pair of primers and at least one second probe, specific for human influenza A/H3 subtypes; and (iii) a third pair of primers and at least one third probe, specific for human influenza B.

In a first embodiment, the first set of primers comprises at least one or more sequences selected from SEQ ID Nos: 1 to 3 or sequences that are at least 80%, 85%, 90% or 95% homologous or identical, or complements of SEQ ID Nos: 1 to 3, and at least one or more sequences selected from SEQ ID Nos: 4 to 7 or sequences that are at least 80%, 85%, 90% or 95% homologous or identical, or complements of SEQ ID Nos: 4 to 7, and preferably the first probe has a sequence selected from SEQ ID Nos: 8 to 10 or a complement thereof, or sequences that are at least 80%, 85%, 90% or 95% homologous or identical, or complements of SEQ ID Nos: 8 to 10 or their complements. In a preferred embodiment, two probes selected from SEQ ID Nos: 8 to 10 or complements thereof, or still more preferred a combination of three probes selected from SEQ ID Nos: 8 to 10 or complements thereof are used according to the invention. It also possible to combine all of the forward primers and all reverse primers and all probes in the methods, kits and compositions according to the present invention.

In a second embodiment, the second set of primers comprises sequences depicted in SEQ ID Nos: 11 and 12 or sequences that are at least 80%, 85%, 90% or 95% homologous or identical, or complements of SEQ ID Nos: 11 and/or 12, and preferably the second probe has a sequence selected from SEQ ID Nos: 13 or 14 or a complement thereof, or sequences that are at least 80%, 85%, 90% or 95% homologous or identical, or complements of SEQ ID Nos: 13 and/or 14. In a preferred embodiment, a combination of two probes having the sequences depicted in SEQ ID Nos: 13 or 14 or complements thereof is used according to the invention.

In a third embodiment, the third set of primers comprises a sequence depicted in SEQ ID No: 15 and at least one of the sequences depicted in SEQ ID Nos: 16 to 19 or sequences that are at least 80%, 85%, 90% or 95% homologous or identical, or complements of SEQ ID Nos: 15 to 19, and preferably the third probe has the sequence of SEQ ID No. 20 or a complement thereof, or sequences that are at least 80%, 85%, 90% or 95% homologous or identical, or complements of SEQ ID No: 20. In a preferred embodiment, a combination of the primer depicted in SEQ ID No: 15 with two, three or four of the primers depicted in SEQ ID Nos: 16 to 19 is possible.

The present invention also relates to a method for the simultaneous detection of influenza A virus (preferably of subtypes H1N1 and/or H3N2) and influenza B virus in a biological sample from a patient, comprising:
a) providing a biological sample from a patient;
b) extracting viral RNA from the biological sample;
c) carrying out a RT-PCR with the set of nucleic acids according to the present invention; and
d) detecting amplification products for each virus, wherein the presence of an amplification product is indicative of the presence of the virus in the biological sample.

In an alternative method, instead of carrying a RT-PCR, the method comprises a step of reverse-transcription and a step of PCR amplification.

The present invention further concerns the use of a set of nucleic acids according to the present invention for simultaneously detecting influenza A and/or influenza B. It further concerns a method of simultaneously detecting influenza A and/or influenza B by using a set of nucleic acids according to the present invention. In addition, it concerns a set of nucleic acids according to the present invention for preparing a diagnostic kit useful for simultaneously detecting influenza A and B. Optionally, the kit further comprises other components such as a DNA polymerase, a reverse-transcriptase, RNase inhibitors, dNTPs and a PCR and/or RT-buffers.

### SHORT SUMMARY OF PREFERRED EMBODIMENTS

Some of the preferred embodiments of the invention are depicted below:
i. A method for the simultaneous detection of the presence or absence of at least one nucleic acid of influenza A subtype H1 and/or H3 and/or influenza B in a biological sample, wherein the method comprises conducting real-time PCR.
ii. The method according to (i) further comprising isolating nucleic acids from the biological sample and performing a reverse transcription step.
iii. The method according to any one of (i) or (ii), wherein the at least one nucleic acid of influenza A subtype H1 and/or H3 and/or influenza B in a biological sample, wherein primer sets that are specific influenza A subtype H1 and/or H3 and/or influenza B are used.
iv. The method according to any one of (i) to (iii), wherein the influenza A subtype H3-specific primer set comprises at least one forward primer and at least reverse primer selected from the oligonucleotide sequences set forth in SEQ ID Nos: 1 to 3 and SEQ ID Nos: 4 to 7 or complements or homologs thereof, or sequences that are at least 80%, 85%, 90% or 95% homologous or identical, or complements of one or more of SEQ ID Nos: 1-7 or complements thereof
v. The method according to any one of (i) to (iii), wherein the influenza A subtype H1-specific primer set comprises at least one forward primer and at least reverse primer selected from the oligonucleotide sequences set forth in SEQ ID NO: 11 and SEQ ID NO: 12 or complements or homologs thereof, or sequences that are at least 80%, 85%, 90% or 95% homologous or identical, or complements of one or more of SEQ ID Nos: 11-12 or complements thereof
vi. The method according to any one of (i) to (iii), wherein the influenza B-specific primer set comprises at least one forward primer and at least reverse primer selected from the oligonucleotide sequences set forth in SEQ ID NO: 15 and SEQ ID Nos: 16 to 19 or complements or homologs thereof, or sequences that are at least 80%, 85%, 90% or 95% homologous or identical, or complements of one or more of SEQ ID Nos: 15-19 or complements thereof
vii. The method according to any one of (i) to (vi), wherein the primer sets selected from the group comprising sequences set forth in SEQ ID Nos: 1 to 3 and 4 to 7 and 11 to 12 and 15 to 19 are used.
viii. The method according to any one of (i) to (vii), wherein at least one probe specifically binding to a nucleic acid of influenza A subtype H1 and/or H3 and/or influenza B is used.
ix. The method according to any one of (i) to (viii), wherein the at least one probe is selected from the oligonucleotides set forth in SEQ ID Nos: 8-10, 13, 14 and/or 20, or sequences that are at least 80%, 85%, 90% or 95% homologous or identical, or complements of one or more of SEQ ID Nos: 1-7 or complements thereof.
x. The method according to any one of (i) to (ix), wherein the primers and or probes carry a fluorescent moiety.
xi. A method for for the diagnosis of an influenza virus infection comprising performing one of the methods according to any one of the preceding embodiments.
xii. A method for monitoring the treatment of influenza virus infection, said method comprising performing the method according to embodiment (xi) before treatment with at least one anti-viral drug and during and/or after treatment with said anti-viral drug.
xiii. An assay for simultaneous detection of at least nucleic acid of influenza A subtype H1 and/or H3 and/or influenza B in a biological sample comprising primers specifically hybridizing to nucleic acids derived from said influenza A subtype H1 and/or H3 and/or influenza B viruses, wherein said assay is suitable for real-time PCR.
xiv. The assay according to (xiii), wherein the assay comprises primers and/or probes set forth in any one of embodiments (iv) to (vi) and (ix).
xv. The assay according to embodiment (xiii) or (xiv), wherein the assay is adapted for use in a fully automated laboratory.
xvi. A diagnostic composition comprising primers and/or probes set forth in any one of embodiments (iv) to (vi) and (ix).
xvii. A diagnostic kit for the simultaneous detection of influenza A subtype H1 and/or H3 and/or influenza B in a biological sample comprising primers and/or probes set forth in any one of embodiments (iv) to (vi) and (ix), and optionally comprising instructions for use.
xviii. The diagnostic kit according to embodiment (xvii), wherein said kit further comprises enzymes, deoxynucleotides, buffers for performing a reverse transcription step and/or a PCR step.
xix. The diagnostic kit according to any one of embodiments (xvii) or (xviii) further comprising reagents for the isolation of nucleic acids from a biological sample.

### SUMMARY

The invention provides for methods of identifying influenza viruses RNA by real-time polymerase chain reaction (PCR) in a biological sample. Primers and probes for detecting influenza are also provided by the invention, as are kits or compositions containing such primers and probes.

Methods of the invention can be used to identify RNA from specimens for diagnosis of influenza infection. The specific primers and probes of the invention that are used in these methods allow for the amplification and monitoring the development of specific amplification products.

In particular a multi-plex assay for seasonal influenza H1, H3 and B subtypes is provided, which allows for simultaneous detection and/or diagnosis of large numbers of different virus subtypes and strains, respectively.

According to one aspect of the invention, a method for detecting the presence or absence of influenza in a biological sample from an individual is provided. As influenza viruses are RNA viruses, the method comprises a reverse transcription step, at least one cycling step, which includes an amplifying step and a hybridizing step. The amplifying step includes contacting the sample with at least one pair of specific primers to produce an amplification product if an influenza nucleic acid molecule is present in the sample. The hybridization step includes contacting the sample with influenza virus specific probes. In the multiplex assays of the present invention several primer pairs are used that are suitable to hybridize to nucleic acids of specific virus subtypes and strains, respectively, but not to other nucleic acids of other subtypes and strains. As a result of the methods described herein, the simultaneous amplification and subsequent detection of the target subtypes and strains is possible. A pair of influenza primers comprises a first influenza primer and a second influenza primer. Sequences of the primers and the probes of the invention are shown in the sequence listing. It is also possible to use more than one forward primer and more than one reverse primer that are specific for the influenza viruses referred to herein in methods, compositions and kits according to the invention.

In some aspects of the invention, the primers and/or probes of the invention can be labeled with a fluorescent moiety. Fluorescent moieties for use in real-time PCR detection are known to persons skilled in the art and are available from various commercial sources, e.g. from life Technologies™ or other suppliers of ingredients for real-time PCR.

Representative biological samples from the respiratory tract include throat swabs, throat washings, nasal swabs, and specimens from the lower respiratory tract.

In addition, the cycling step can be performed on a control sample. A control sample can include the same portion of the influenza nucleic acid molecule. Alternatively, a control sample can include a nucleic acid molecule other than an influenza nucleic acid molecule.

Cycling steps can be performed on such a control sample using a pair of control primers and a pair of control probes. The control primers and probes are different from influenza primers and probes.

One or more amplifying steps produces a control amplification product. Each of the control probes hybridizes to the control amplification product.

In another aspect of the invention, there are provided articles of manufacture, or kits.

Kits of the invention can include at least one pair of specific primers for the amplification of influenza virus A H1 and/or H3 and influenza virus B and at least one influenza probe hybridizing specifically with the amplification products. It is however possible to include more than one, e.g. two, three or four forward and reverse primers, respectively, or to include all of the forward primers and reverse primers and all of the probes disclosed in the sequence listing.

Articles of manufacture can include fluorophoric moieties for labeling the primers or probes or the primers and probes are already labeled with donor and corresponding acceptor fluorescent moieties.

The article of manufacture can also include a package insert having instructions thereon for using the primers, probes, and fluorophoric moieties to detect the presence or absence of influenza in a sample.

In another aspect of the invention, there is provided a method for detecting the presence or absence of influenza in a biological sample from an individual. Such a method includes performing at least one cycling step. A cycling step include at least one amplifying step and a hybridizing step. Generally, an amplifying step includes contacting the sample with a pair of primers to produce an amplification product if an influenza nucleic acid molecule is present in the sample. Generally, a hybridizing step includes contacting the sample with an influenza-specific probe. The probe is usually labeled with at least one fluorescent moiety. The presence or absence of fluorescence is indicative of the presence or absence of influenza in said sample.

Amplification generally involve the use of a polymerase enzyme. Suitable enzymes are known in the art, e.g. Taq Polymerase, etc.

In another aspect of the invention, there is provided a method for detecting the presence or absence of influenza in a biological sample from an individual. Such a method includes performing at least one cycling step. A cycling step can include an amplifying step and a dye-binding step. An amplifying step generally includes contacting the sample with a pair of influenza-specific primers to produce an influenza amplification product if an influenza nucleic acid molecule is present in the sample. A dye-binding step generally includes contacting the influenza amplification product with a double-stranded DNA binding dye. The method further includes detecting the presence or absence of binding of the double-stranded DNA binding dye into the amplification product. According to the invention, the presence of binding is typically indicative of the presence of influenza nucleic acid in the sample, and the absence of binding is typically indicative of the absence of influenza nucleic acid in the sample. Such a method can further include the steps of determining the melting temperature between the amplification product and the double-stranded DNA binding dye. Generally, the melting temperature confirms the presence or absence of influenza nucleic acid. Representative double-stranded DNA binding dyes include SYBRGREEN I®, SYBRGOLD®, and ethidium bromide.

In another aspect, the invention allows for the use of the methods described herein to determine whether or not an individual is in need of treatment for influenza.

Treatment for influenza can include, e.g., administration of a neuraminidase inhibitor (e.g., oseltamivir phosphate) to the individual.

The invention also provides for the use of the articles of manufacture described herein to determine whether or not an individual is in need of treatment for influenza.

Further, the methods and/or the articles of manufacture described herein can be used to monitor an individual for the effectiveness of a treatment for influenza as well as in epidemiology to monitor the transmission and progression of influenza from individuals to individuals in a population. The methods and/or the articles of manufacture (e.g., kits) disclosed herein can be used to determine whether or not a patient is in need of treatment for influenza.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will be decisive.

The details of one or more embodiments of the invention are set forth in the description below. Other features, objects, and advantages of the invention will be apparent from the description, and from the claims.

### DETAILED DESCRIPTION

According to the present invention, a real-time PCR assay for detecting influenza virus nucleic in a biological sample that is more sensitive and specific than existing assays is described herein.

Primers and probes for detecting influenza infections and articles of manufacture containing such primers and probes are also provided. The increased sensitivity of real-time PCR for detection of influenza as well as the improved features of real-time PCR including sample containment and real-time detection of the amplified product, make feasible the implementation of this technology for routine diagnosis of influenza infections in the clinical laboratory.

The invention provides methods to detect influenza by amplifying, for example, a portion of an influenza nucleic acid derived from influenza A subtypes H1 and/or H3 and from influenza B. Nucleic acid sequences from influenza A are available, e.g. in the Influenza Sequence Database (ISD) (flu.lanl.gov on the World Wide Web, described in Macken et al., 2001, “The value of a database in surveillance and vaccine selection” in Options for the Control of Influenza IV. A.D.M.E., Osterhaus & Hampson (Eds.), Elsevier Science, Amsterdam, pp. 103-106).

Primers and probes can be designed using, for example, a computer program such as OLIGO (Molecular Biology Insights, Inc., Cascade, Colo.). Important features when designing oligonucleotides to be used as amplification primers include, but are not limited to, an appropriate size amplification product to facilitate detection, similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 15 to 30 nucleotides in length. Designing oligonucleotides to be used as hybridization probes can be performed in a manner similar to the design of primers, although the members of a pair of probes preferably anneal to an amplification product. As with oligonucleotide primers, oligonucleotide probes usually have similar melting temperatures, and the length of each probe must be sufficient for sequence-specific hybridization to occur but not so long that fidelity is reduced during synthesis. Oligonucleotide probes are generally 15 to 30 nucleotides in length. Primers useful within the context of the present invention include oligonucleotides suitable in PCR reactions for the amplification of nucleic acids derived from influenza A and B subtypes and strains, respectively.

In describing and claiming the present invention, the terminology and definitions hereinbelow are used for the purpose of describing particular embodiments only, and are not intended to be limiting.

As used in this specification and the appended claims, the singular forms “a,” “an,” and “the” include plural referents unless the context clearly dictates otherwise. The term “multiplex” refers to multiple assays that are carried out simultaneously, in which detection and analysis steps are generally performed in parallel. As used herein, a multiplex assay may also be an assay that is suitable to simultaneously amplify and identify different target nucleic acids of one particular influenza virus subtype or strain.

Within the context of the present invention, a multiplex assay would be for example, a molecular assay that simultaneously screens for influenza A (preferably H1 and/or H3 subtypes) and influenza B.

As used herein, the term “probe” or “detection probe” refers to an oligonucleotide that forms a hybrid structure with a target sequence contained in a molecule (i.e., a “target molecule”) in a sample undergoing analysis, due to complementarity of at least one sequence in the probe with the target sequence. The nucleotides of any particular probe may be deoxyribonucleotides, ribonucleotides, and/or synthetic nucleotide analogs.

The term “primer” or “amplification primer” refers to an oligonucleotide that is capable of acting as a point of initiation for the 5′ to 3′ synthesis of a primer extension product that is complementary to a nucleic acid strand. The primer extension product is synthesized in the presence of appropriate nucleotides and an agent for polymerization such as a DNA polymerase in an appropriate buffer and at a suitable temperature.

As used herein, the term “homology” means that a sequence, e.g. a primer or probe sequence disclosed herein, is essentially identical to the sequence of said primer or probe, but may instead of deoxyribunucleotides comprise corresponding ribonucleotides or synthetic analogues. Homologs of a given sequence hybridize to the same target sequence and permit amplification of a target region in a gene of interest, or they bind to target regions as probes and may be detected, e.g. because they carry fluorescent moieties. Identical sequences correspond to the sequences of the primers and/or probes of the present invention, but they may not be 100% identical, e.g. when one or more residues of the total sequences have been replaced by another residue, or because the 5′ or 3′ ends of the primers/probes disclosed herein have been shortened or lengthened. Such primers/probes maintain their capability of hybridizing with a target region and permitting amplification or detection of said target region.

As used herein, the term “target amplification” refers to enzyme-mediated procedures that are capable of producing billions of copies of nucleic acid target. Examples of enzyme-mediated target amplification procedures known in the art include PCR.

Within the context of the present invention, the nucleic acid “target” is the nucleic acid sequence of Influenza A and/or Influenza B, preferably of Influenza A subtypes H1 and/or H3 and/or Influenza B.

The most widely used target amplification procedure is PCR, first described for the amplification of DNA by Mullis et al. in U.S. Pat. No. 4,683,195 and Mullis in U.S. Pat. No. 4,683,202 and is well known to those of ordinary skill in the art. Where the starting material for the PCR reaction is RNA, complementary DNA (“cDNA”) is made from RNA via reverse transcription. A PCR used to amplify RNA products is referred to as reverse transcriptase PCR or “RT-PCR.” In the PCR technique, a sample of DNA is mixed in a solution with a molar excess of at least two oligonucleotide primers of that are prepared to be complementary to the 3′ end of each strand of the DNA duplex; a molar excess of nucleotide bases (i.e., dNTPs); and a heat stable DNA polymerase, (preferably Taq polymerase), which catalyzes the formation of DNA from the oligonucleotide primers and dNTPs. Of the primers, at least one is a forward primer that will bind in the 5′ to 3′ direction to the 3′ end of one strand of the denatured DNA analyte and another is a reverse primer that will bind in the 3′ to 5′ direction to the 5′ end of the other strand of the denatured DNA analyte. The solution is heated to 94-96° C. to denature the double-stranded DNA to single-stranded DNA. When the solution cools down and reaches the so-called annealing temperature, the primers bind to separated strands and the DNA polymerase catalyzes a new strand of analyte by joining the dNTPs to the primers. When the process is repeated and the extension products synthesized from the primers are separated from their complements, each extension product serves as a template for a complementary extension product synthesized from the other primer. As the sequence being amplified doubles after each cycle, a theoretical amplification of a huge number of copies may be attained after repeating the process for a few hours; accordingly, extremely small quantities of DNA may be amplified using PCR in a relatively short period of time.

Where the starting material for the PCR reaction is RNA, as in the case of Influenza virus nucleic acids, complementary DNA (“cDNA”) is synthesized from RNA via reverse transcription. The resultant cDNA is then amplified using the PCR protocol described above. Reverse transcriptases are known to those of ordinary skill in the art as enzymes found in retroviruses that can synthesize complementary single strands of DNA from an mRNA sequence as a template. A PCR used to amplify RNA products is referred to as reverse transcriptase PCR or “RT-PCR.”

The terms “real-time PCR” and “real-time RT-PCR,” refer to the detection of PCR products via a fluorescent signal generated by the coupling of a fluorogenic dye molecule and a quencher moiety to the same or different oligonucleotide substrates. Examples of commonly used probes are TAQMAN® probes, Molecular Beacon probes, SCORPION® probes, and SYBR® Green probes. Briefly, TAQMAN® probes, Molecular Beacons, and SCORPION® probes each have a fluorescent reporter dye (also called a “fluor”) attached to the 5′ end of the probes and a quencher moiety coupled to the 3′ end of the probes. In the not hybridized state, the proximity of the fluor and the quencher molecules prevents the detection of fluorescent signal from the probe; during PCR, when the polymerase replicates a template on which a probe is bound, the 5′-nuclease activity of the polymerase cleaves the probe thus, increasing fluorescence with each replication cycle. SYBR Green® probes binds double-stranded DNA and upon excitation emit light; thus as PCR product accumulates, fluorescence increases. In the context of the present invention, the use of TAQMAN® probes is preferred.

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C. Within the context of the present invention, it is to be understood that the specific sequence lengths listed are illustrative and not limiting and that sequences covering the same map positions, but having slightly fewer or greater numbers of bases are deemed to be equivalents of the sequences and fall within the scope of the invention, provided they will hybridize to the same positions on the target as the listed sequences. Because it is understood that nucleic acids do not require complete complementarity in order to hybridize, the probe and primer sequences disclosed herein may be modified to some extent without loss of utility as specific primers and probes. Sequences of primers or probes that are at least 80%, 85%, 90% or 95% homologous or identical, or complements of one or more of the primers of the invention or of complements thereof fall also in the scope of the present invention. As is known in the art, hybridization of complementary and partially complementary nucleic acid sequences may be obtained by adjustment of the hybridization conditions to increase or decrease stringency, i.e., by adjustment of hybridization temperature or salt content of the buffer.

The term “hybridizing conditions” is intended to mean those conditions of time, temperature, and pH, and the necessary amounts and concentrations of reactants and reagents, sufficient to allow at least a portion of complementary sequences to anneal with each other. As is well known in the art, the time, temperature, and pH conditions required to accomplish hybridization depend on the size of the oligonucleotide probe or primer to be hybridized, the degree of complementarity between the oligonucleotide probe or primer and the target, and the presence of other materials in the hybridization reaction admixture. The actual conditions necessary for each hybridization step are well known in the art or can be determined without undue experimentation.

The term “label” as used herein refers to any atom or molecule that can be used to provide a detectable (preferably quantifiable) signal, and that can be attached to a nucleic acid or protein via a covalent bond or noncovalent interaction (e.g., through ionic or hydrogen bonding, or via immobilization, adsorption, or the like). Labels generally provide signals detectable by fluorescence, chemiluminescence, radioactivity, colorimetry, mass spectrometry, X-ray diffraction or absorption, magnetism, enzymatic activity, or the like. Examples of labels include fluorophores, chromophores, radioactive atoms, electron-dense reagents, enzymes, and ligands having specific binding partners.

As used herein, the term “sample” as used in its broadest sense to refer to any biological sample from any human or veterinary subject that may be tested for the presence or absence of one or more influenza virus specific nucleic acids, preferably nucleic acids of Influenza A, e.g. of subtypes H1 and/or H3 and/or Influenza B. The samples may include, without limitation, tissues obtained from any organ, such as for example, lung tissue; and fluids obtained from any organ such as for example, blood, plasma, serum, lymphatic fluid, synovial fluid, cerebrospinal fluid, amniotic fluid, amniotic cord blood, tears, saliva, and nasopharyngeal washes.

The term “patient” as used herein is meant to include both human and veterinary patients.

The amplification primers and detection probes of the present invention are set forth in the sequence listing.

In one aspect of the invention, there is provided a method for detection of Influenza A and/or B, preferably of Influenza A subtypes H1 and/or H3 and/or of Influenza B in a sample comprising the steps of obtaining a biological sample from a patient; isolating nucleic acid from the sample; amplifying the nucleic acid, wherein the nucleic acid is amplified and detected with amplification primers and detection probes selected from the group depicted in the sequence listing.

In another aspect of the invention, there is provided a method for detection of Influenza A and/or B, preferably of Influenza A subtypes H1 and/or H3 and/or of Influenza B in a sample comprising the steps of obtaining a tissue sample from a patient; extracting nucleic acids from the sample; amplifying the nucleic acid, wherein the RNA is amplified and detected with amplification primers and detection probes as depicted in the sequence listing.

In one embodiment of the invention, the nucleic acid is selected from RNA and DNA. When the nucleic acid is RNA, it is amplified using real time RT-PCR. When the nucleic acid is DNA, it is amplified using real time PCR.

In another embodiment of the invention, the sample is a tissue fluid from a human or animal patient, which may be selected from the group consisting of blood, plasma, serum, lymphatic fluid, synovial fluid, cerebrospinal fluid, amniotic fluid, amniotic cord blood, tears, saliva, and nasopharyngeal washes.

In another embodiment of the invention, the assay is a component of a devices that is suitable in fully automated laboratories capable of extracting nucleic acids from a sample (e.g. using the epMotion System of Eppendorf International), optionally capable of reverse transcribing isolated nucleic acids, performing amplification reactions using the assay components described herein and quantitatively and qualitatively detecting nucleic acid targets, e.g. using real-time PCR.

In a further aspect, the present invention relates to a composition comprising any of the above mentioned primers and probes. Preferably, the composition comprises also ingredients, e.g. enzymes, buffers and deoxynucleotides necessary for reverse transcription and/or PCR, preferably for qualitative and/or quantitative RT-PCR. The composition may be stored in the refrigerator in a liquid state or deep-frozen in a suitable medium, or it may be lyophilized and reconstituted before use and which may further comprises detectable probes and/or an internal control.

The present invention further provides a kit comprising the assay of the invention and optionally instructions for use.

It is to be understood that while the invention has been described in conjunction with the embodiments described herein, that the foregoing description as well as the examples that follow are intended to illustrate and not limit the scope of the invention. Other aspects, advantages and modifications within the scope of the invention will be apparent to those skilled in the art to which the invention pertains. All patents and publications mentioned herein are incorporated by reference in their entireties.

The following examples are put forth so as to provide those of ordinary skill in the art with a complete disclosure and description of how to make and use the compositions of the invention. The examples are intended as non-limiting examples of the invention. While efforts have been made to ensure accuracy with respect to variables such as amounts, temperature, etc., experimental error and deviations should be taken into account. Unless indicated otherwise, parts are parts by weight, temperature is degrees centigrade, and pressure is at or near atmospheric. All components were obtained commercially unless otherwise indicated.

### EXAMPLES

### Example 1

### Seasonal Influenza Sub-Typing Kit-SH1N1/H3N2/B

RNA of influenza viruses grown in culture was amplified using primers specific for Influenza A seasonal H1N1 (PR8), Influenza B and Influenza A-H3N2 (H17) and detected using specific probes. In detail, a primer mixture comprising primer sets for seasonal-H1N1, H3N2 and Influenza B was used, wherein the concentration of each primer is 200 nM. The Influenza B Taqman® probe is labeled with ROX reporter fluorophore and BHQ-2 quencher. The Influenza A H3N2 Taqman® probe is labeled with FAM reporter fluorophore and BHQ-1 quencher. The seasonal H1N1 Taqman® probe is labeled with JOE reporter fluorophore and BHQ-1 quencher. QuantiTect Multiplex RT-PCR NR Kit is supplied by Qiagen company and used for reverse transcription and PCR amplification in accordance with the manufacturer's instructions.

Each RT-PCR reaction is performed in triplicate. RNA templates are reverse transcribed into cDNA for 20 min at 50° C. followed by a denaturation step at 94° C. for 15 minutes. Each of the 45 amplification cycles consisted of 94° C. for 45 seconds and 60° C. for 45 seconds.

Results and Discussion

Influenza A-H1N1 amplification was detected in the yellow channel using the Rotor-gene Q real-time PCR cycler (Qiagen). Influenza B was detected in the orange channel and Influenza A-H3N2 was detected in the green channel. A signal is considered positive when the CT value is below 40. The experiments showed that the assay functions over a wide range concentrations of the same template.

TABLE 1A

| | | Replicate 1 | Replicate 2 | Replicate 3 |
|---|---|---|---|---|
| | unit | (CT) | (CT) | (CT) |
| H1N1(PR8) | | | | |
| 3.5 | pg | 22.75 | 23.22 | 23.05 |
| 350 | fg | 26.79 | 26.04 | 26.11 |
| 35 | fg | 30.46 | 30.62 | 29.79 |
| H3N2(H17) | | | | |
| 8.23 | pg | 22.94 | 23.74 | 22.62 |
| 823 | fg | 26.44 | 26.57 | 25.8 |
| 82.3 | fg | 30.35 | 29.71 | 30.47 |
| FluB | | | | |
| 22.15 | pg | 17.34 | 17.14 | 17.1 |
| 2.215 | pg | 20.65 | 20.48 | 20.49 |
| 221.5 | fg | 23.86 | 23.74 | 23.47 |
| 22.15 | fg | 27.59 | 27.19 | 27.19 |
| 2.215 | fg | 30.97 | 30.98 | 31.31 |
| Negative | | x | x | x |
| control (H₂O) | | | | |

Design of Influenza A Seasonal H1N1, H3N2 and Influenza B Primers and Probes

Primers and probes for the detection of seasonal H1N1 and H3N2, respectively, were designed on the basis of sequence alignments to detect a conserved nucleotide region of the hemagglutinin protein (HA region) found in different viral isolates from sources all over the world.

Similarly, primers and probes for the detection of Influenza B were designed on the basis of sequence alignments to detect a conserved nucleotide region of the Matrix gene (M gene) found in different viral isolates from sources all over the world.

Specific Primers and Probes Combination for Seasonal H1N1:

TABLE 1B

| | | |
|---|---|---|
| Forward | 011 | TCCCATCCATTCAATCCAGAGG |
| Forward | 012 | TCCCATCAATTCAATCAAGAGG |
| Forward | 013 | TCCCATCCGTTCAATCTAGAGG |
| Forward | 014 | TCCCATCCGTTCAATCCAGAGG |
| Reverse | 011 | ACCCAGATCCTTGCTCATTCTG |
| Reverse | 012 | AGCCAGATCCTTGCTCATTCTG |
| Probe | 011 | CCTTCAATGAAACCGGCAATGGCTCC |
| Probe | 012 | CCCTTCAATGAAACCAGCAATGGCTCC |

Primer and Probes with Degenerate Nucleotides for Seasonal H1N1:

TABLE 1C

| | | |
|---|---|---|
| S-H1N1 SET B F | F10 | TCCCATCCATTCAATCHAGAGG |
| S-H1N1 SET B R | R10 | ASCCAGATCCTTGCTCATTCTG |

H and S are degenerate nucleotides, wherein S=G or C; H=A, T, or C)

TABLE 1D

| | | S-H1N1/H3/B | | | | | |
|---|---|---|---|---|---|---|---|
| Template | | all primers + probes | | | S-H1N1/H3/B | | |
| H1N1 | | (Specific primers | | | all primers + probes | | |
| (PR8) | | combined) | | | (Degenerate nucleotides) | | |
| RNA | unit | #1 (CT) | #2 (CT) | #3 (CT) | #1 (CT) | #2 (CT) | #3 (CT) |
| 35 | pg | 19.89 | 20.19 | 19.37 | 21.8 | 21.82 | 20.67 |
| 3.5 | pg | 22.91 | 23.51 | 23.04 | 24.45 | 23.77 | 23.46 |
| 350 | fg | 26.85 | 27.55 | 27.65 | 27.53 | 27.58 | 27.37 |
| | | x | x | x | x | x | x |

The primers and probes are capable of detecting sequences that are 90% homologous with respect to the H1N1 (A/PR/8/34).

It has surprisingly been found that the multi-plex assay of the present invention is highly sensitive as it was possible to detect as low as 100 copies of the viral genome.

The multi-plex assay using three sets of primers and probes and one set of primer and probe for the extraction control is surprisingly as efficient as a single-plex assay, wherein only one set of primers and probes for one specific template is used, i.e. mixing three sets of primer and probes did not result in a loss of sensitivity during amplification of the templates. This means that the present invention provides a highly sensitive and fast assay that can be used in diagnosis and determination of the source of a suspected influenza infection.

### Example 2

### Test of Inventive Seasonal Influenza Kit

The Influenza H1N1, H3N2, B RT-PCR Test constitutes a ready-to-use system for the detection of seasonal influenza viruses (including subtypes of H1N1, H3N2 and B) RNA using polymerase chain reaction (PCR).

Tests were run on the Rotor-Gene Q MDX 5plex HRM instrument. The PCR mix used contain reagents and enzymes for the specific amplification of small size fragments of RNA targeting on Hemagglutinin gene or Matrix protein gene, and for the direct detection of the specific amplicons in the Green, Yellow and Orange fluorescence channels of the Rotor-Gene Q MDX 5plex HRM for H3N2, H1N1 and B, respectively. In addition, a second set of primers/probes was used that was designed to detect an extraction control target in the Red fluorescence channel. This extraction control, present allows to assess the efficiency of extraction and acts as an RT-PCR reaction control. This second amplification system does not reduce the detection limit of the analytical Influenza viral RNA PCR. The test also comprises a positive control (Influenza sub-typing PC) and a negative control (RNA NC) that allow to assess that the PCR reaction performed properly.

The test was used for the qualitative detection and differentiation of seasonal Influenza H1N1 (excluding pandemic A(H1N1)2009 virus), H3N2 and Influenza B in nasal swabs from human patients with signs and symptoms of respiratory tract infections.

Analytical Sensitivity

The “analytical sensitivity” refers to the limit of detection. The limit of detection (LoD) is defined as the lowest concentration of analyte that can be consistently detected (in ≧95% of samples tested under routine clinical laboratory conditions in a defined type of specimen). The LoD was determined on three representative strains, which are A/FM/1/47(H1N1), A/Victoria/3/75(H3N2) and B/Hong Kong/5/72. The LoD of each representative strain had been identified, and shown in the following Table 2.

TABLE 2

| Strain | Subtype | LoD (CEID50/mL) |
|---|---|---|
| A/FM/1/47(H1N1) | H1N1 (flu A) | 6.76 × 10² |
| A/Victoria/3/75 (H3N2) | H3N2 (flu A) | 3.95 × 10¹ |
| B/Hong Kong /5/72 | Influenza B | 3.95 × 10⁰ |

Analytical Reactivity and Specificity

The analytical reactivity test is to determine whether the assay can detect additional clinically relevant seasonal Influenza H1N1 (excluding pandemic A(H1N1)2009 virus), H3N2 and Influenza B strains representing temporal and geographical diversity. The specificity test is to identify cross-reactivity with other microorganisms which may be present in clinical samples from patient respiratory tract. It could be shown that many influenza strains were detected except for four H1N1 strains, which are very old influenza strains. The hemagglutinin nucleotide sequences (target sequences) of these strains have been analyzed, and the homogeneity is very low compared to the circulating strain,

A/Brisbane/59/2007(H1N1), which is one of the vaccine candidate strains for seasonal influenza H1N1. The potential cross-reactivity of the Sentosa SA Seasonal H1N1, H3N2, B RT-PCR Test was assessed using the control group listed in Table 7.2. None of the tested pathogens were reactive, confirming the specificity of the Test for influenza seasonal viruses (cf. Tables 3 and 4)

TABLE 3
Summary of test results for analytical reactivity

| | | | | | Extraction |
|---|---|---|---|---|---|
| | | H3N2 | H1N1 | Flu B | control |
| | | detection | detection | detection | detection |
| | | (Green | (Yellow | (Orange | (Red |
| Virus, strain | Source | channel) | channel) | channel) | channel) |
| A/FM/1/47 (H1N1) | ATCC | − | + | − | + |
| A/Denver/1/57 (H1N1) | ATCC | − | − | − | + |
| A/Weiss/43 (H1N1) | ATCC | − | − | − | + |
| A/NWS/33 (H1N1) | ATCC | − | − | − | + |
| A/New Jersey/8/76 (H1N1) | ATCC | − | − | − | + |
| A/Brisbane/59/2007 (H1N1) | Vircell | − | + | − | + |
| A/Christchurch/1/2003 (H1N1) | NIBSC | − | + | − | + |
| A/Aichi/2/68 (H3N2) | ATCC | + | − | − | + |
| A/Victoria/3/75 (H3N2) | ATCC | + | − | − | + |
| A/Hong Kong/8/68 (H3N2) | ATCC | + | − | − | + |
| A/Port Chalmers/1/73 (H3N2) | ATCC | + | − | − | + |
| A/Perth/16/2009 (H3N2) | Vircell | + | − | − | + |
| B/Lee/40 | ATCC | − | − | + | + |
| B/Maryland/1/59 | ATCC | − | − | + | + |
| B/Taiwan/2/62 | ATCC | − | − | + | + |
| B/Allen/45 | ATCC | − | − | + | + |
| B/GL/1739/54 | ATCC | − | − | + | + |
| B/Hong Kong/5/72 | ATCC | − | − | + | + |
| B/Brisbane/60/2008 | Vircell | − | − | + | + |
| B/Jiangsu/10/2003 | NIBSC | − | − | + | + |

TABLE 4
Summary of test results for analytical specificity

| | H3N2 | H1N1 | Flu B | Extraction |
|---|---|---|---|---|
| | detection | detection | detection | control (EC) |
| | (Green | (Yellow | (Orange | detection |
| Tested pathogens (strain) | channel) | channel) | channel) | (Red channel) |
| Human Parainfluenza virus 2 (Greer) | − | − | − | + |
| Human Parainfluenza virus 3 (C243) | − | − | − | + |
| Human Respiratory Syncytial virus (B1 wt), | − | − | − | + |
| BWV/14617/85 | | | | |
| Measles virus (Edmonston) | − | − | − | + |
| Mumps, Jones | − | − | − | + |
| Human Coronavirus (229E) | − | − | − | + |
| Human Coronavirus (OC43) | − | − | − | + |
| Enterovirus, strain H | − | − | − | + |
| Enterovirus type 71 (BrCt) | − | − | − | + |
| Human Parainfluenza virus 1 (C-35) | − | − | − | + |
| Human Metapneumovirus | − | − | − | + |
| Adenovirus Type 7 (Gomen) | − | − | − | + |
| Adenovirus 1 (Adenoid 71) | − | − | − | + |
| Cytomegalovirus (huHerpesvirus 5), (Davis) | − | − | − | + |
| Epstein Barr virus (P-3) | − | − | − | + |
| Bordetella Pertussis | − | − | − | + |
| Mycobacterium tuberculosis (H37Ra) | − | − | − | + |
| Pseudomonas aeruginosa | − | − | − | + |
| (Boston 41501) | | | | |
| Moraxella catarrhalis (20) | − | − | − | + |
| Streptococcus pyogenes Rosenbach | − | − | − | + |
| Neisseria Meningitidis, serotype B | − | − | − | + |
| Straphylococcus aureus (TCH1516) | − | − | − | + |
| Corynebacterium diphtheria (NCTC 13129) | − | − | − | + |
| Streptococcus salivarius (DSMN 13084) | − | − | − | + |
| Haemophilus influenza (ATCC 51907) | − | − | − | + |
| Neisseria Subflava | − | − | − | + |
| Legionella pneumophila (Philadelphia-1) | − | − | − | + |
| Escherichia coli, serotype O103 | − | − | − | + |
| Lactobacillus acidophilus (ATCC 314) | − | − | − | + |
| Straphylococcus epidermidis, FDA | − | − | − | + |
| (PCI 1200) | | | | |
| Streptococcus pneumonia (CIP 104225) | − | − | − | + |
| Mycoplasma pneumonia (ATCC 29342) | − | − | − | + |
| Chlamydophila puemoniae (TW-183) | − | − | − | + |

Reproducibility

The inter/intra-assay reproducibility was determined by testing 3 concentrations of RNA in duplicates together with a negative control (RNA NC), a positive control (Influenza sub-typing PC) and water (NTC). The overall reproducibility assessment setup allows to test intra-assay variability (variability of multiple results of samples of the same concentration within one experiment), the inter-assay variability (variability of multiple results of the assay generated on different instruments of the same type by different operators within one laboratory) and the inter-batch variability (variability of multiple results of the assay using various batches of mastermix and primer/probe mix). There are three different influenza subtype targets, i.e. H1N1, H3N2, B, and one representative strain from ATCC are tested at three different concentrations for each subtype target. Tests consistently gave >99% reproducibility.

Copy Number Determination

The test is to quantify virus pathogens by using positive control RNA to convert the existing virus titer such as CEID50/mL into copy number unit (copies/reaction). The virus with defined titer unit such as CEID50/mL first went through sample extraction process. Then 5 μL of elute (out of 100 μL) go through the inventive test manually along with positive control (PC) RNA standard curve for one-step PCR. The serial dilution of PC (standard curve) is defined in copy numbers (copies/mL). This will justify conversion of the LoD of virus between CEID50/mL unit and copy number unit.

The test was done on representative strains including A/FM/1/47(H1N1), A/Victoria/3/75 (H3N2) and B/Hong Kong/5/72. The equivalent amounts at two different units are shown in the following Table 5.

TABLE 5

| | Concentration at | Concentration at |
|---|---|---|
| Strain | CEID₅₀/mL | copies/reaction |
| A/FM/1/47(H1N1) | 3.38 × 10⁵ | 6.59 × 10⁴ |
| A/Victoria/3/75 (H3N2) | 1.58 × 10⁴ | 3.74 × 10⁴ |
| B/Hong Kong/5/72 | 1.58 × 10³ | 2.49 × 10⁴ |

Measurement Range

The Limit of Detection (LoD) of the representative influenza virus stocks has been identified above. The tested measurement range for influenza virus is summarized as the following Table 6.

TABLE 6

| | | Tested Highest | |
|---|---|---|---|
| | | Titer | LoD |
| Strain | Subtype | (CEID50/mL) | (CEID50/mL) |
| A/FM/1/47(H1N1) | H1N1 | 3.38 × 106 | 6.76 × 102 |
| | (flu A) | | |
| A/Victoria/3/75(H3N2) | H3N2 | 1.58 × 105 | 3.95 × 101 |
| | (flu A) | | |
| B/Hong Kong/5/72 | Influenza | 1.58 × 104 | 3.95 × 100 |
| | B | | |

In addition, the PC is tested in quantification of copy number. From the standard curve, the dynamic range for the assay is ˜10² to 10⁷ copies per reaction, or ˜10⁴ copies/mL to 10⁹ copies/mL in general for clinical samples.

Clinical Sensitivity and Specificity

The test is established based on retrospective samples. Nasal swab samples were collected from individuals who were symptomatic with influenza-like illness (ILI). All samples were subjected to nucleic acid extraction and detection by the inventive H1N1, H3N2, B RT-PCR test. A total of 280 clinical specimens have been tested by the Sentosa SA Influenza Seasonal H1N1, H3N2, B RT-PCR Test, and the clinical sensitivity and clinical specificity are 93.5% and 98.4% respectively as shown in Table 7.

TABLE 7
2 × 2 Counts

| Test Result | Negative | Positive | Total Subjects |
|---|---|---|---|
| Absent | 245 | 2 | 247 |
| Present | 4 | 29 | 33 |
| -All- | 249 | 31 | 280 |

Sensitivity=93.5%; Specificity=98.4%; PPV=87.9%; NPV=99.2%

Stability—Storage Stability

Two tests were performed to evaluate storage stability of the components: accelerated stability study and real-time stability study. In accelerated stability study, estimated stability=Accelerated stability×2^{ΔT/10}, where ΔT is the difference between temperature of accelerated stability storage and actual storage temperature. If 37° C. accelerated stability is used to estimate stability of analytes stored in −20° C., this gives a factor of 52. In this example, samples stable until 1 week in 37° C. is stable for 52 weeks in −20° C. (ABI Whitepaper: Product Stability Study—TaqMan® Gene Expression Assays). Real-time stability involves keeping the reagents at the designated storage temperature for the duration of time tested. Accelerated stability study for the inventive test shows that the performance of the kit is stable for at least 0.5 years when stored in room temperature. Accelerated stability study shows that inventive test is stable for 1 year when stored in −20° C.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for the simultaneous detection of the presence or absence of at least one nucleic acid of influenza A subtype H1 and/or H3 and/or influenza B in a biological sample, wherein the method comprises:
(a) isolating nucleic acids from the biological sample and optionally performing a reverse transcription step,
(b) conducting real-time PCR, wherein primer sets that are specific for nucleic acids of influenza A subtype H1 and/or H3 and/or influenza B are used,
wherein the influenza A subtype H3-specific primer set comprises oligonucleotide sequences set forth in SEQ ID NOs: 1 to 3 and SEQ ID NOs: 4 to 7, or complements thereof, or sequences that are at least 90% or 95% homologous or identical, or complements of one or more of SEQ ID NOs: 1-7 or complements thereof, and
wherein the influenza A subtype H1-specific primer set comprises oligonucleotide sequences set forth in SEQ ID NO: 11 and SEQ ID NO: 12, or complements thereof, or sequences that are at least 90% or 95% homologous or identical, or complements of one or more of SEQ ID NOs: 11-12 or complements thereof, and
wherein the influenza B-specific primer set comprises oligonucleotide sequences set forth in SEQ ID NO: 15 and SEQ ID NOs: 16 to 19, or complements thereof, or sequences that are at least 90% or 95% homologous or identical, or complements of one or more of SEQ ID NOs: 15-19 or complements thereof, and
wherein at least one probe specifically binding to a nucleic acid of influenza A subtype H1 and/or H3 and/or influenza B is used, and said at least one probe is selected from the oligonucleotides set forth in SEQ ID NOs: 8 to 10, 13, 14, and 20, or complements thereof, or sequences that are at least 90% or 95% homologous or identical, or complements of one or more of SEQ ID NOs: 8-10, 13, 14, and 20, or complements thereof.

2. The method according to claim 1, wherein the primers and/or probes carry a fluorescent moiety.

3. An in vitro method for the diagnosis of an influenza virus infection in a subject comprising performing the method according to claim 1.

4. A method for monitoring the treatment of influenza virus infection, said method comprising performing the method according to claim 3 before treatment with at least one anti-viral drug and during and/or after treatment with said anti-viral drug.

5. A real-time PCR assay for the simultaneous detection of at least one nucleic acid of influenza A subtype H1 and/or H3 and/or influenza B in a biological sample comprising primers and/or probes having oligonucleotide sequences as set forth in claim 1.

6. The assay according to claim 5, wherein the assay is adapted for use in a fully automated laboratory.

7. A composition comprising primers and/or probes having oligonucleotide sequences as set forth in claim 1.

8. A kit for the simultaneous detection of influenza A subtype H1 and/or H3 and/or influenza B in a biological sample comprising primers and/or probes having oligonucleotide sequences as set forth in claim 1 and instructions for use.

9. The kit according to claim 8, wherein said kit further comprises enzymes, deoxynucleotides, and/or buffers for performing a reverse transcription step and/or a PCR step.

10. The diagnostic kit according to claim 8 further comprising reagents for the isolation of nucleic acids from a biological sample.


SEQUENCE LISTING (23 sequences)

SEQ ID NO: 1 - DNA, 24 bp, Influenza A virus
tgatcagattgaagtgactaatgc

SEQ ID NO: 2 - DNA, 24 bp, Influenza A virus
tgaccaaattgaagtgactaatgc

SEQ ID NO: 3 - DNA, 24 bp, Influenza A virus
tgaccaaattgaagttactaatgc

SEQ ID NO: 4 - DNA, 18 bp, Influenza A virus
ggaagccatcacactgag

SEQ ID NO: 5 - DNA, 18 bp, Influenza A virus
ggaagccatcacaatgag

SEQ ID NO: 6 - DNA, 20 bp, Influenza A virus
ggaagtcatcacaatgaggg

SEQ ID NO: 7 - DNA, 20 bp, Influenza A virus
gaaaaacatcacaatgaggg

SEQ ID NO: 8 - DNA, 24 bp, Influenza A virus
ctggttcagagttcctcaacaggt

SEQ ID NO: 9 - DNA, 24 bp, Influenza A virus
ctggtccagagttcctcaacaggt

SEQ ID NO: 10 - DNA, 24 bp, Influenza A virus
ctagttcagagctcctcaacgggg

SEQ ID NO: 11 - DNA, 22 bp, Influenza A virus
tcccatccattcaatchagagg

SEQ ID NO: 12 - DNA, 22 bp, Influenza A virus
asccagatccttgctcattctg

SEQ ID NO: 13 - DNA, 26 bp, Influenza A virus
ccttcaatgaaaccggcaatggctcc

SEQ ID NO: 14 - DNA, 26 bp, Influenza A virus
cccttcaatgaaaccagcaatggctc

SEQ ID NO: 15 - DNA, 19 bp, Influenza B virus
caattgcctacctgctttc

SEQ ID NO: 16 - DNA, 19 bp, Influenza B virus
ggtcaaactctttcccacc

SEQ ID NO: 17 - DNA, 19 bp, Influenza B virus
ggtcaaattctttcccacc

SEQ ID NO: 18 - DNA, 19 bp, Influenza B virus
agtcaaattctttcccacc

SEQ ID NO: 19 - DNA, 19 bp, Influenza B virus
gatcaaattctttcccacc

SEQ ID NO: 20 - DNA, 25 bp, Influenza B virus
cagaagatggagaaggcaaagcaga

SEQ ID NO: 21 - DNA, 18 bp, Tobacco mosaic virus
gacgtgcgaattcctcag

SEQ ID NO: 22 - DNA, 21 bp, Tobacco mosaic virus
gttgatgttgcatactggttg

SEQ ID NO: 23 - DNA, 25 bp, Tobacco mosaic virus
aacctcttgaactgacagcgtgtgc